(19) United States
(12) Patent Application Publication
(10) Pub. No.: US 2024/0399074 A1
(43) Pub. Date: Dec. 5, 2024

(54) PRESSURE TRANSDUCER FOR INJECTIONS

(71) Applicant: Ray King, Houston, TX (US)

(72) Inventor: Ray King, Houston, TX (US)

(21) Appl. No.: 18/723,540

(22) PCT Filed: Dec. 23, 2022
(86) PCT No.: PCT/US2022/082338

§ 371 Date: Jun. 24, 2024

Related U.S. Application Data
(60) Provisional application No. 63/293,238, filed on Dec. 23, 2021.

(51) Int. Cl. A61M 5/48; A61M 5/31; A61M 5/315

(52) U.S. Cl. CPC A61M 5/488; A61M 5/3134; A61M 5/315; A61M 2205/0216; A61M 2205/3341

(57) ABSTRACT

The present invention relates to methods and systems for accurately measuring the injection pressure of a filler into the arterial vessels of the body for functional and/or cosmetic procedures, and maintaining this injection pressure below a desired pressure. For example, the injection pressure may be kept below a specific patient's diastolic blood pressure, hence reducing or negating the incidence of function-threatening embolism into a subsequent artery due to retrograde substance injection. A pressure transducer may be desirable to intrinsically control the injection pressure in response to the applied pressure on the syringe (e.g. via a plunger or other actuator). Ideally, a device (e.g. specifically tailored with properties to a specific material being injected) which consistently limits the injection pressure to being lower than an intended value is desirable, such as through intrinsic response by a pressure transducer.

### CROSS-REFERENCE TO RELATED APPLICATIONS

This application is a 371 national stage application of Patent Cooperation Treaty patent application Ser. No. PCT/US2022/082338, which claims the benefit and priority of U.S. provisional patent application Ser. No. 63/293,238. The contents of the foregoing applications are hereby incorporated by reference in their entireties.

### FIELD OF THE INVENTION

This invention relates generally to methods and systems for the safe injection of functional and/or cosmetic substance into the vessels of the body, such as to help reduce or obviate the risk of complications (e.g. stroke or blindness) by retrograde injection of the injected substance into another undesired artery, particularly to methods and systems utilizing pressure-mediated flow control in a syringe.

### BACKGROUND OF THE INVENTION

Present-day injection of a substance for functional and/or cosmetic procedures has a non-negligible risk of retrograde injection, and when injected into an arteriovenous malformation, aneurysm neck, or the face as a filler, can be pushed retrograde into an artery, subsequently filling another artery that then causes an embolism to occlude a key function-supply artery (e.g. causing stroke or compromising vision).

A method that relies upon subjective “slow” injection is oblivious to the actual key force to overcome—the worst-case diastolic blood pressure in a given patient.

### SUMMARY OF THE INVENTION

The present invention relates to methods and systems for accurately measuring the injection pressure of a filler into the arterial vessels of the body for functional and/or cosmetic procedures, and maintaining this injection pressure below a desired pressure. For example, the injection pressure may be kept below a specific patient's diastolic blood pressure, hence reducing or negating the incidence of function-threatening embolism into a subsequent artery due to retrograde substance injection. A pressure transducer may be desirable to intrinsically control the injection pressure in response to the applied pressure on the syringe (e.g. via a plunger or other actuator). Ideally, a device (e.g. specifically tailored with properties to a specific material being injected) which consistently limits the injection pressure to being lower than an intended value is desirable, such as through intrinsic response by a pressure transducer.

In one aspect of the invention, a pressure transducer may be placed to be in contact or fluid communication with both the reservoir and the lumen of a needle attached to a syringe such that pressure changes in the reservoir cause the pressure transducer to alter the flow characteristics of a fluid entering the lumen of the needle from the reservoir. In some exemplary embodiments, the pressure transducer may include a flexible membrane which is adapted to deform due to pressure in the reservoir and constrict or otherwise change the size of an entry into the lumen of the needle attached to a syringe. In some embodiments, the flexible membrane may generally constrict or otherwise reduce the size of the entry into the lumen of the needle attached to a syringe in response to an increased or high amount of pressure in the reservoir to, for example, restrict the flow through the lumen. This may be desirable to reduce the effective pressure of the fluid exiting the needle due to the decreased flow. For example, a flexible membrane may form a concentric channel that connects to the lumen entrance and may collapse inward to shrink the size of the channel in response to increased pressure in the reservoir. In response to a decreased or lower level of pressure in the reservoir, the flexible membrane may then flex back or relax to increase or return the channel size to enable higher flow into the lumen through the larger channel.

In another aspect of the invention, the reservoir, pressure transducer and entrance to the lumen of a needle in a syringe may be configured such that pressure applied to a fluid in the reservoir causes the fluid to flow past or otherwise contact to exert pressure on the pressure transducer prior to entering the entrance to the lumen. In some embodiments, a channel or other flow guide (or multiple) may be provided to direct flow of a fluid in the reservoir being pressurized, such as by a plunger or other actuator, such that the entrance of the lumen is placed downstream from the pressure transducer and the plunger or other actuator does not directly pressurize the fluid at the entrance of the lumen without first passing by or contacting the pressure transducer. This may be useful in aiding the pressure transducer in acting on the entrance to the lumen due to fluid pressure prior to the fluid being able to enter the lumen for upstream intrinsic control of the flow into the lumen.

The present invention together with the above and other advantages may best be understood from the following detailed description of the embodiments of the invention illustrated in the drawings.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIG. 1 illustrates a schematic view of a syringe and needle with a pressure transducer with lower pressure being applied and a flexible membrane unconstructed to enable flow into the needle; and

FIG. 2 illustrates a schematic view of a syringe and needle with a pressure transducer with higher pressure being applied and a flexible membrane being constricted to decrease flow into the needle.

### DETAILED DESCRIPTION OF THE INVENTION

The detailed description set forth below is intended as a description of the presently exemplified methods, devices and compositions provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods and systems for accurately measuring the injection pressure of a filler into the arterial vessels of the body for functional and/or cosmetic procedures, and maintaining this injection pressure below a desired pressure. For example, the injection pressure may be kept below a specific patient's diastolic blood pressure, hence reducing or negating the incidence of function-threatening embolism into a subsequent artery due to retrograde substance injection. A pressure transducer may be desirable to intrinsically control the injection pressure in response to the applied pressure on the syringe (e.g. via a plunger or other actuator). Ideally, a device (e.g. specifically tailored with properties to a specific material being injected) which consistently limits the injection pressure to being lower than an intended value is desirable, such as through intrinsic response by a pressure transducer. This may be useful to reduce or obviate the incidence of retrograde embolus from the injected substance, as by maintaining this injection pressure below a given value for the diastolic blood pressure measured in the patient, the risk of such embolism would be negated. Such embolisms could subsequently travel down another artery branch via which an embolus could compromise key function (e.g. vision) or cause other problems, such as a stroke.

In a typical surgical setting, the diastolic blood pressure of a given patient may be measured such that a syringe with the proper pressure transducer may be selected, in an effort to control injection pressure to limit it to staying below diastolic pressure by utilizing a pressure transducer that limits injection pressure to an appropriate level. Other injection devices may generally be utilized in place of a syringe/needle as appropriate or desired in connection with the descriptions below.

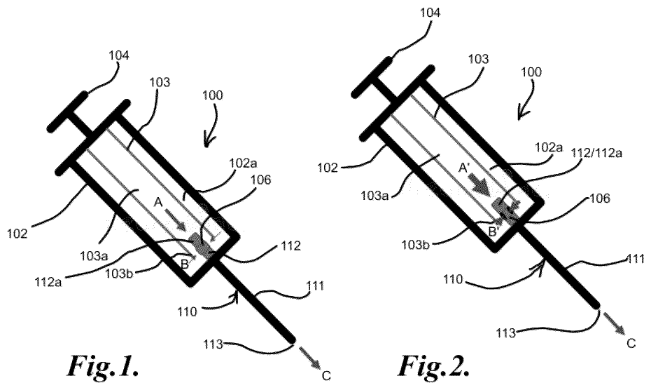

In one aspect of the invention, an embodiment of which is illustrated in the schematic views of FIGS. 1 and 2, a pressure transducer 106 may be placed approximately between (or at the interface between) the body 102 of a syringe 100 (which may generally include the reservoir 102a for a substance to be injected) and an injection portion, such as a needle 110 with an internal lumen 111, an entrance portion 112 (such as illustrated with a tube portion with a channel that connects to the internal lumen 111) and an injection aperture 113. The pressure transducer 106 may be placed to be in contact or fluid communication with both the reservoir 102a and the lumen 111 of a needle 110 attached to a syringe 100 such that pressure changes in the reservoir 102a cause the pressure transducer 106 to alter the flow characteristics of a fluid entering the lumen 111 of the needle 110 from the reservoir 102a. The fluid in the reservoir 102a may generally be pressurized to cause flow out of the syringe 100, such as by applying pressure with an actuator 104, as illustrated with plunger 104, which may, for example, be pressed into at least a portion of the reservoir 102a to decrease its volume to cause fluid to flow into the entry 112 for the lumen 111.

In some exemplary embodiments, the pressure transducer 106 may include a flexible membrane, as illustrated, which is adapted to deform due to pressure in the reservoir 102a and constrict or otherwise change the size of an entry 112 into the lumen 111 of the needle 110 attached to a syringe 100. The material, dimensions, thickness, and/or other physical parameters of the flexible membrane may generally be selected to have the proper response to the fluid pressure in the reservoir 102a so as to deform in a manner as described.

In some embodiments, the flexible membrane may generally constrict or otherwise reduce the size of the entry 112 into the lumen 111 of the needle 110 attached to a syringe 100 in response to an increased or high amount of pressure in the reservoir 102a to, for example, restrict the flow through the lumen 111. This may be desirable to reduce the effective pressure of the fluid exiting C the needle 110 through aperture 113 due to the decreased flow. For example, a flexible membrane may form a concentric channel that connects to the lumen entrance 112 and may collapse inward to shrink the size of the channel in response to increased pressure in the reservoir. The concentric channel may form a part of the channel in the entry 112, such as illustrated with the pressure transducer 106 placed in the middle portion of the entry 112 with section 112a extending from it. In response to a decreased or lower level of pressure in the reservoir 102a, the flexible membrane may then flex back or relax to increase the channel size to enable higher flow into the lumen 111 through the larger channel.

In general, the material parameters of the flexible membrane may be such that the flexible membrane does not significantly deform when pressure in the reservoir 102a is below a desired level, which would enable unobstructed flow of a fluid into the entry 112, as shown in FIG. 1 with low pressure B on the pressure transducer 106 with the desired flow A. The material may also generally be elastic such that it will intrinsically resist deformation and return substantially to its original form when the pressure in the reservoir is decreased or removed. It may also be desirable for the material parameters to be configured to provide a proportional response to the pressure in the reservoir 102a, such as, for example, to provide at least some level of metered response to differing applied pressures to generate a relatively constant or otherwise controlled flow out of the aperture 113. In general, the pressure transducer 106 may include one, or multiple flexible membranes which may be identical or have differing parameters to modulate the overall response of the pressure transducer 106. As discussed below, the flexible membrane may refer to a single membrane or to multiple membranes interchangeably.

Above that desired level, as shown in FIG. 2 with increased pressure B′, the material would deform to an extent that the entry 112 is constricted or partially obstructed by the flexible membrane's deformation to lower the flow A′ into the entry 112, such that in both cases the flow C out of the needle aperture 113 remains at relatively the same pressure or at/below a desired pressure level (i.e. by the pressure being applied to the flexible membrane, the membrane being displaced to constrict the entry 112 to the lumen 111 would narrow the area for flow, hence reducing flow around that area and limiting the amount of flow exiting per a given surface area (hence controlling pressure, since pressure is flow/area)).

In another aspect of the invention, the reservoir 102a, pressure transducer 106 and entrance 112 to the lumen 111 of a needle 110 in a syringe 100 may be configured such that pressure applied to a fluid in the reservoir 102a causes the fluid to flow past or otherwise contact to exert pressure on the pressure transducer 106 prior to entering the entrance 112 to the lumen 111. In some embodiments, a channel or other flow guide (or multiple) may be provided to direct flow of a fluid in the reservoir 102a being pressurized, such as by a plunger 104 or other actuator, such that the entrance 112 of the lumen 111 is placed downstream from the pressure transducer 106 and the plunger 104 or other actuator does not directly pressurize the fluid at the entrance 112 of the lumen 111 without first passing by or contacting the pressure transducer 106. This may be useful in aiding the pressure transducer 106 in acting on the entrance 112 to the lumen 111 due to fluid pressure prior to the fluid being able to enter the lumen 111 for upstream intrinsic control of the flow into the lumen 111. FIGS. 1 and 2 illustrate a channel guide 103 which forms a substantially concentric cylindrical tube portion with the entry 112. In this configuration, the plunger 104 or other actuator would directly act on or pressurize only the portion of the reservoir 102a outside of the channel guide 103 such that fluid would be forced to flow into the internal space 103a of the channel guide 103 through the open end 103b which is situated at or before the pressure transducer 106. The fluid would then act on the pressure transducer 106 prior to entering the internal space 103a before reaching the entry 112 and flowing into the lumen 111 of the needle 110.

In some embodiments, the flow characteristics between the channel guide 103 and the reservoir 102a may be configured to alter the pressure change of the fluid between the reservoir 102a and the aperture 113. For example, the section 112a may be configured to alter the flow distance required for the fluid to reach the lumen 111 after entering the internal space 103a, such as, for example, by lengthening the section 112a into the internal space 103a to increase the time the fluid is present in the internal space 103a before entering the entry 112. This may, for example, provide extra resistance to flow to decrease the pressure of the fluid. The size of the gap between the open end 103b and the pressure transducer 106 may also be configured to alter the flow characteristics, such as by narrowing the gap to cause a larger portion of the fluid pressure to be exerted on the pressure transducer 106 before entering the internal space 103a, which may, for example, cause a smaller increase in pressure to cause a greater deformation of the flexible membrane than with a larger gap.

### Example of Filler Administration Using Presently Described Intrinsically Controlled Pressure Regulator Mechanism

A syringe pre-filled with filler shall be obtained (shown with syringe 100), with an inside partition circumferentially built-in (shown with channel guide 103), along with an inner tube (shown with entry 112) continuous with the exit lumen (111) made of rigid plastic material with an intervening flexible membrane (shown with pressure transducer 106) comprising a portion of the distal wall (just proximal to the lumen exit)—see FIGS. 1 and 2. When pressure is applied, if the pressure does not deform the flexible membrane diaphragm (or does so nominally) the lumen shall retain its diameter without much compromise, and allow delivery of flow accordingly. However, if high pressure is applied to the syringe plunger, then it shall translate throughout the filler material, putting pressure on the flexible membranes resulting in lumen narrowing, limiting flow—the greater the pressure, the greater the luminal narrowing (along the adluminal side) and the reduction in flow. Hence the amount of filler injection distally will be limited, and be of controlled pressure, utilizing such mechanism, helping avoid adverse events such as high amount of filler extruded at high pressure.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A system for controlling injection pressure of an injection device comprising:
an injection device having a substance reservoir and an injection portion having an entry portion with an internal channel;
an actuator adapted to pressurize said substance reservoir; and
a pressure transducer disposed between said substance reservoir and said injection portion and adapted to deform in response to pressure in said substance reservoir to alter said entry portion to said injection device.

2. The system of claim 1, wherein said actuator is a plunger adapted to pressurize said substance reservoir.

3. The system of claim 1, wherein said injection portion comprises a needle.

4. The system of claim 1, wherein said pressure transducer comprises a flexible membrane disposed concentrically with said internal channel of said entry portion.

5. The system of claim 1, further comprising a channel guide having a substantially cylindrical form disposed substantially concentrically with said entry portion and having an open end that is positioned about said pressure transducer.

6. The system of claim 1, wherein said entry portion comprises an extension section extending from said pressure transducer into said channel guide.
